(19) United States
(12) Patent Application Publication
(10) Pub. No.: US 2021/0338894 A1
(43) Pub. Date: Nov. 4, 2021

(54) Artificial Periosteum

(71) Applicant: Orthocell Limited, MURDOCH, W.A. (AU)

(72) Inventor: Ming-Hao Zheng, City Beach (AU)

(21) Appl. No.: 17/275,110

(22) PCT Filed: Sep. 13, 2019
(86) PCT No.: PCT/AU2019/050984

§ 371 Date: Mar. 10, 2021

(30) Foreign Application Priority Data
Sep. 14, 2018 (AU) 2018903480

(51) Int. Cl. A61L 27/46; A61L 27/54; A61K 38/18

(52) U.S. Cl. CPC A61L 27/46; A61L 27/54; A61K 38/1875; A61L 2300/416; A61L 2300/404; A61L 2300/414; A61L 2300/41; A61L 2430/02

(57) ABSTRACT

The invention relates to an artificial periosteum comprising a functionalized collagen-containing membrane and a drug-carrier mixture, wherein the drug-carrier mixture comprises at least one therapeutic agent and a calcium-containing carrier mixture. The invention also relates to use of the artificial periosteum for repairing bone.

### FIELD

The present invention relates to an artificial periosteum, systems, and methods for repair of bone and the use of the artificial periosteum to locally deliver therapeutic agents such as bone active agents.

### BACKGROUND

Many medical procedures today rely on regenerating bone, which has deteriorated as a result of a disease or age or has been damaged (e.g., fractured). While a variety of surgical procedures are available, the advancement of modern medicine has allowed for certain techniques to augment, and sometimes even substitute for these surgeries.

Periosteum is the connective tissue that surrounds bones, has the capacity to regenerate both cartilage and bone. This unique tissue contains two discrete layers: the inner cambium layer which is believed to contain the undifferentiated mesenchymal stem cells responsible for fracture repair and the outer fibrous layer. Periosteum has been used successfully in biological resurfacing for the repair of damaged articular cartilage. For deep osteochondral defects, a bone graft can be used to replace the damaged subchondral bone. However, potential problems with the use of bone grafts include obtaining grafts of the appropriate size and shape, graft-site morbidity, and tissue integration with the surrounding tissue.

The development of an artificial periosteum with the biochemical and mechanical properties of an autologous osteochondral graft with better integration properties and without the need for osteochondral graft harvesting would be very attractive.

A further benefit of an artificial periosteum is that it could also be used to successfully deliver therapeutic agents such as bone active drugs like BMP-2 for cortical bone regeneration.

The existing approved material used for delivery of recombinant human BMP-2 (rhBMP-2) is a porous collagen sponge approved by the FDA. Other carrier materials for rhBMP-2 have been described in the literature as well (Morales et al., (2017), J Drug Delivery Sciences and Technology, Vol 42). The current issue with the approved biomaterial is its rapid degradation which leads to a burst release of the protein and a secondary pro-osteoclast effect which reduces the overall net bone formation. Moreover, porous biomaterials are used for overall bone regeneration by delivering rhBMP-2, but Horstmann and co-workers have reported that these materials tend to protrude into the cortical bone and delay cortical healing (Horstmann et al. (2018), Tissue Eng. Part A, Vol. 23). Thus, from a clinical perspective there is a need to have a membrane in the form of a thin biomaterial that can prevent cancellous bone void fillers from protruding into the cortical bone and simultaneously promote the natural course of cortical bone healing by providing a template for cells and local release of growth factors. The process of cancellous bone healing is different than cortical bone healing and this invention is primarily intended for cortical bone regeneration. The cancellous bone can be treated with any bone substitute but the cortical bone requires specific biomaterial properties.

Thus, there remains a need for improved bone repair methods especially the development of an artificial periosteum which could also be used to deliver bone active agents like BMP-2 locally over extended periods of time to promote bone growth and repair bone defects.

### SUMMARY

The present invention provides an artificial periosteum and methods related to bone repair and local delivery of therapeutic agents into a bone. The therapeutic agents may be bone active agents which repair bone defects and otherwise promote bone growth, bone active agents that treat bone-related pain, anti-inflammatory agents to treat inflammation-related conditions (e.g., arthritis), anti-cancer drugs to treat bone cancer, or antimicrobial agents to treat or prevent infection at the treatment site or combinations thereof.

One aspect of the invention provides an artificial periosteum comprising a functionalized collagen-containing membrane and a drug-carrier mixture, wherein the drug-carrier mixture comprises at least one therapeutic agent and a calcium-containing carrier mixture.

Another aspect of the invention provides a method of repairing bone, comprising the step of implanting an artificial periosteum comprising a functionalized collagen-containing membrane and a drug-carrier mixture, wherein the drug-carrier mixture comprises at least one therapeutic agent and a calcium-containing carrier mixture.

Also disclosed is an artificial periosteum comprising a functionalized collagen-containing membrane and a drug-carrier mixture for use in a method of repairing bone, wherein the drug-carrier mixture comprises at least one therapeutic agent and a calcium-containing carrier mixture.

In certain embodiments of the invention, the functionalized collagen-containing membrane is a hydroxyapatite functionalized collagen-containing membrane, while the calcium-containing carrier mixture is collagen based.

Thus, in one aspect the present invention provides an artificial periosteum comprising a hydroxyapatite functionalized collagen-containing membrane and a drug-carrier mixture, wherein the drug-carrier mixture comprises BMP-2 and zoledronic acid (ZA).

In certain embodiments of the invention, the therapeutic agent is a bone active agent which comprises a compound that activates osteoblasts. In other embodiments, the therapeutic agent is a bone active agent that inhibits osteoclasts. In still other embodiments, the therapeutic agent is a bone active agent which comprises one or more of: PGE1; PGE2; an EP2 receptor agonist; an EP4 receptor agonist; an EP2 receptor/EP4 receptor dual agonist; an organic bisphosphonate; a cathepsin K inhibitor; an estrogen or an estrogen receptor modulator; calcitonin; an inhibitor of osteoclast proton ATPase; an inhibitor of HMG-CoA reductase; an integrin receptor antagonist; a RANKL inhibitor; a bone anabolic agent; a bone morphogenetic agent; Vitamin D or a synthetic Vitamin D analogue; an androgen or an androgen receptor modulator; a SOST inhibitor; platelet-derived growth factor; a pharmaceutically acceptable salt thereof; and a mixture thereof.

One aspect of the invention provides a method of repairing bone, comprising the step of implanting an artificial periosteum comprising a functionalized collagen-containing membrane and a drug-carrier mixture, wherein the drug-carrier mixture comprises BMP-2 and zoledronic acid.

In one embodiment, the functionalized collagen-containing membrane is a hydroxyapatite functionalized collagen-containing membrane.

Also disclosed is an artificial periosteum comprising a functionalized collagen-containing membrane and a drug-carrier mixture for use in a method of repairing bone in a patient, wherein the drug-carrier mixture comprises BMP-2 and zoledronic acid

In one aspect of the invention provides a method of repairing a bone defect in a patient, wherein the method comprises the step of:
(i) implanting into said bone defect a bone graft material; and
(ii) covering said graft with a hydroxyapatite functionalized collagen-containing membrane.

Also disclosed is a bone graft material covered with a hydroxyapatite functionalized collagen-containing membrane for use in a method of repairing a bone defect in a patient.

### BRIEF DESCRIPTION OF THE FIGURES

FIG. 1 shows an overview of the material structure and collagen fiber alignment.

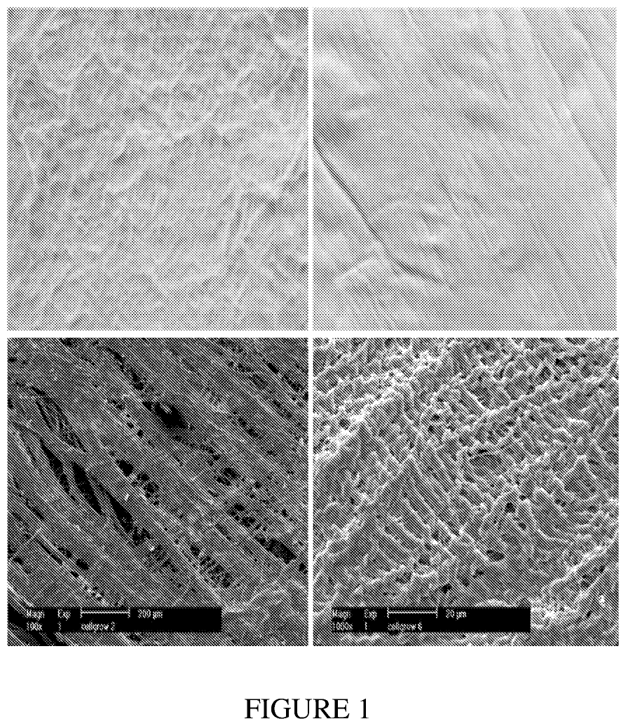

FIG. 2 shows a surgical procedure of the tibia defect model.

FIG. 3 shows micro-CT quantifications of the tibia defect study 8-weeks post-surgery.

FIG. 4 shows the evaluation of cortical healing using micro-CT.

FIG. 5 shows the histological analysis of tibia defect healing.

FIG. 6 shows X-Ray radiography of specimens harvested from the abdominal muscle pouch 4 weeks post-surgery.

FIG. 7 shows the role of collagen membrane as a containment device for ceramic or polymeric biomaterials placed within a bone void.

FIG. 8 shows a comparative study of an absorbable collagen sponge (ACS) produced by Medtronic, which is sold as INFUSE® bone graft, together with solution containing rhBMP-2, with the collagen-containing membrane of the present invention comprising rhBMP-2 and ZA. Data are CT data and represent average ±SD (shown at top), n=8/group for ACS and n=5/group for collagen membrane.

### DETAILED DESCRIPTION

It is well understood that in the repair of a bone defect caused by trauma, infection or tumour, it is normal to replace the missing/removed material with allo-, auto- or synthetic graft material. If the bone defect involves a cortical loss it takes significant time to build a new cortex, even if healing has taken place in the cancellous bone. Depending on the size of the cortical defect, and especially if it is segmental, it might not heal. Same is the case with fractures of non-unions, which comprise up to 5% of all high impact fractures.

Periosteum is suspected to be involved in the successful healing of bone defects since periosteal cells have a strong role in cortical bone healing. A special procedure for critical defects is where a spacer is temporarily inserted to create a soft tissue shell (becoming highly metabolically active) around the spacer resembling a periosteum and then a bone transplantation is carried out removing the spacer after a few months. The temporary periosteum so formed is resutured and the graft is allowed to heal into normal bone.

The artificial periosteum of the present invention is an ideal material to repair bone defects as the collagen-containing membrane acts as a cover for the bone defect and in some aspects also provides the replacement material. The artificial periosteum of the present invention reduces bulging, leakage and when functionalized with biomolecules, forms a new bridging cortex. It may be glued, sutured or knotted with a circumferential loop to or around bone. It may be applied with on lay or in lay techniques inserted under the cortex. If functionalized with bone active agent, it will speed up cortical bone regeneration.

In the broadest aspect, the present invention relates to a collagen-containing membrane that has been functionalized.

The term “collagen” as used herein refers to all forms of collagen, including those which have been processed or otherwise modified. Preferred collagens are treated to remove the immunogenic telopeptide regions (“atelopeptide collagen”), are soluble, and will have been reconstituted into fibrillar form.

The term “collagen-containing membrane” refers to a piece or segment of collagen-containing tissue that has been produced by methods known in the art and disclosed, for example, in U.S. Pat. No. 7,096,688. The collagen-containing membrane can be any geometric shape but is typically substantially planar and may, in position, conform to the shape of underlying or overlying tissue.

The collagen-containing membrane preferably has the following properties:
a) pores that interconnect in such a way as to favour tissue integration and vascularisation;
b) biodegradability and/or bioresorbability so that normal tissue ultimately replaces the collagen-containing membrane;
c) surface chemistry that promotes cell attachment, proliferation and differentiation;
d) strength and flexibility; and
e) low antigenicity.

The collagen-containing membrane is typically prepared or manufactured from “collagen-containing tissue” comprising dense connective tissue found in any mammal. The term “collagen-containing tissue” means skin, muscle and the like which can be isolated from a mammalian body that contains collagen. The term “collagen-containing tissue” also encompasses “synthetically” produced tissue in which collagen or collagen containing material has been assembled or manufactured outside a body.

In some embodiments, the collagen-containing tissue is isolated from a mammalian animal including, but not limited to, a sheep, a cow, a pig or a human. In other embodiments, the collagen-containing tissue is isolated from a human.

In some embodiments, the collagen-containing tissue is “autologous”, i.e. isolated from the body of the patient in need of treatment.

In some embodiments, the collagen-containing membrane will comprise greater than 80% type I collagen. In other embodiments, the collagen-containing membrane will comprise at least 85% type I collagen. In still other embodiments the collagen-containing membrane will comprise greater than 90% type I collagen.

The collagen-containing membrane may be manufactured by any method known in the art; however, one preferred method includes the following steps:
(i) isolating a collagen-containing tissue and incubating the tissue in an ethanol solution;
(ii) incubating the collagen-containing tissue from step (i) in a first solution comprising an inorganic salt and an anionic surfactant in order to denature non-collagenous proteins contained therein;
(iii) incubating the collagen-containing tissue produced in step (ii) in a second solution comprising an inorganic acid until the collagen in said material is denatured; and
(iv) incubating the collagen-containing tissue produced in step (iii) in a third solution comprising an inorganic acid with simultaneous mechanical stimulation for sufficient time to enable the collagen bundles in said collagen-containing tissue to align; wherein the mechanical stimulation comprises applying tension cyclically to the collagen-containing tissue.

It will be appreciated that any inorganic salt may be used in the first solution as long as it is capable of forming a complex with Lewis acids. In some embodiments, the inorganic salt is selected from the group consisting of trimethylammonium chloride, tetramethylammonium chloride, sodium chloride, lithium chloride, perchlorate and trifluoromethanesulfonate. In other embodiments, the inorganic salt is lithium chloride (LiCl).

While any number of anionic surfactants may be used in the first solution, in some embodiments, the anionic surfactant is selected from the group consisting of alkyl sulfates, alkyl ether sulfates, alkyl sulfonates, and alkyl aryl sulfonates. Particularly useful anionic surfactants include alkyl sulphates such as sodium dodecyl sulphate (SDS).

In some embodiments, the first solution comprises about 1% (v/v) SDS and about 0.2% (v/v) LiCl.

In some embodiments, the inorganic acid in the second solution comprises about 0.5% (v/v) HCl, while the inorganic acid in the third solution comprises about 1% (v/v) HCl.

It will be appreciated by those skilled in the art that the incubation periods in each of the three steps will vary depending upon: (i) the type of collagen-containing tissue; (ii) the type of inorganic salt/acid and/or anionic surfactant; (iii) the strength (concentration) of each inorganic salt/acid and/or anionic surfactant used and (iv) the temperature of incubation. In some embodiments, the incubation period in step (i) is at least 8 hours. In other embodiments, the incubation period in step (ii) is less than 60 minutes, while in other embodiments the incubation period in step (iii) is at least 20 hours.

In some embodiments, the incubation in step (ii) is at about 4° C. In other embodiments, the incubation in step (ii) is undertaken for at least 12 hours.

In some embodiments, the second solution comprises about 0.5% (v/v) HCl.

In some embodiments, the incubation in step (iii) is undertaken for about 30 minutes. In other embodiments, the incubation in step (iii) is undertaken with shaking.

In some embodiments, the third solution comprises about 1% (v/v) HCl solution.

In some embodiments, the incubation in step (iv) is undertaken for about 12 to 36 hours, preferably for about 24 hours. In other embodiments, the incubation in step (iv) is undertaken with shaking.

In some embodiments, the method further comprises a neutralization step between step (iii) and step (iv) which comprises incubation of said collagen-containing tissue with about 0.5% (v/v) NaOH.

In some embodiments, the methods further comprises step (v) which comprises incubating the collagen-containing tissue from step (iv) with acetone and then drying the collagen-containing tissue.

In some embodiments, the method further comprises between steps (ii) and (iii) and/or between steps (iii) and (iv) a step of contacting the collagen-containing tissue with glycerol in order to visualise and facilitate the removal of fat and/or blood vessels.

The glycerol maybe contacted with the collagen-containing tissue for any amount of time that will facilitate the removal of fat and/or blood vessels. In some embodiments, the contact time is at least 10 minutes.

In some embodiments, the method further comprises between steps (ii) and (iii) and/or between steps (iii) and (iv) a wash step for the collagen-containing tissue. The purpose of the wash step used between steps (ii) and (iii) is to remove denatured proteins. Thus, any wash solution capable of removing denatured proteins can be used. In some embodiments the wash solution used between steps (ii) and (iii) is acetone.

Following the washing with acetone, the collagen-containing tissue is further washed with sterile water.

In some embodiments, the collagen-containing tissue is further washed in a NaOH:NaCl solution. If the collagen-containing tissue is washed with NaOH:NaCl it is then preferably washed with sterile water.

In some embodiments, after step (iv) the collagen-containing tissue is further washed with the first solution.

The term “simultaneous mechanical stimulation” used in the methods described herein refers to the process of stretching the collagen-containing tissue during the chemical processing of the collagen-containing tissue. The collagen-containing tissue may undergo static and/or cyclic stretching. Accordingly, in some embodiments the simultaneous mechanical stimulation may comprise:
(i) stretching of the collagen-containing tissue for a preset period;
(ii) relaxation of the collagen-containing tissue for a preset period; and
(iii) n-fold repetition of steps (i) and (ii), where n is an integer greater than or equal to 1.

If the mechanical stimulation is carried out by stretching the collagen-containing tissue, the collagen-containing tissue is preferably stretched along its long axis.

In some embodiments, the simultaneous mechanical stimulation comprises applying tension cyclically to collagen-containing tissue, wherein the periodicity of the tension comprises a stretching period of about 10 seconds to about 20 seconds and a relaxing period of about 10 seconds, and the strain resulting therefrom is approximately 10%, and the mechanical stimulation continues until the collagen bundles within the collagen-containing tissue are aligned as described herein.

Once produced the collagen-containing tissue comprises collagen fibres or bundles with a knitted structure. The term “knitted structure” as used herein refers to a structure comprising first and second groups of fibres or bundles where fibres or bundles in the first group extend predominately in a first direction and fibres or bundles in the second group extend predominately in a second direction, where the first and second directions are different to each other and the fibres or bundles in the first group interleave or otherwise weave with the fibres or bundles in the second group. The difference in direction may be about 90°.

The collagen-containing tissue made by the preferred methods comprise a “maximum tensile load strength” of greater than 20N. In some embodiments, the collagen-containing tissue of the present invention has maximum tensile load strength greater than 25N, 40N, 60N, 80N, 100N, 120N or 140N.

Further, it is believed that the knitted structure of the embodiments of the collagen-containing tissue provides reduced extension at maximum load of the collagen-containing patch while providing an increase in modulus.

The term “modulus” as used herein means Young's Modulus and is determined as the ratio between stress and strain. This provides a measure of the stiffness of the collagen-containing tissue and/or patch.

In some embodiments the collagen-containing tissue has a modulus of greater than 100 MPa. In other embodiments the collagen-containing tissue has a modulus of greater than 200 MPa, 300 MPa, 400 MPa, or 500 MPa.

The term “extension at maximum load” as used herein means the extension of the collagen-containing tissue at the maximum tensile load strength referenced to the original length of the collagen-containing tissue in a non-loaded condition. This is to be contrast with maximum extension which will be greater.

In some embodiments, the collagen-containing tissue has extension at maximum load of less than 85% of the original length.

Once the collagen-containing tissue has been produced it may then be shaped into a collagen-containing membrane for use. In some embodiments, the collagen-containing membrane is adapted by shaping the membrane to provide better means of manipulation in situ.

Preferably, the collagen-containing membrane of the present invention is sufficiently thick to provide support for the drug-carrier mixture; however, not too thick that the ability to manipulate the collagen-containing membrane in situ is impaired. Thus, in some embodiments the collagen-containing membrane is between 25 μm and 200 μm thick. In some embodiments, the collagen-containing membrane is between 30 μm and 180 μm thick. In other embodiments, the collagen-containing membrane is between 35 μm and 170 μm thick. In still other embodiments, the collagen-containing membrane is between 40 μm and 160 μm thick. In still other embodiments, the collagen-containing membrane is between 45 μm and 150 μm thick. In still other embodiments, the collagen-containing membrane is between 50 μm and 140 μm thick. In still other embodiments, the collagen-containing membrane is between 50 μm and 100 μm thick. Finally, in some embodiments the collagen-containing membrane is about 50 μm thick.

One form of the collagen-containing membrane sees the membrane being perforated to allow transport of native bone active molecules or therapeutically-active agents in the graft material to be able to pass on and through the membrane to recruit circulating stem cells and pericytes from the overlaying muscle.

The collagen-containing membrane preferably possesses two distinct surfaces (one either side): a smooth surface featuring compact collagen bundles and a rough, porous surface of loose collagen fibres. The rough side is especially good at promoting cell attachment and in practice, when the membrane is being used with an overlaying muscle, it is crucial that the rough side is facing the muscle. However, in cases where there is no overlaying muscle, such as in the repair of distal tibia, it is less critical that the rough side faces any particular surface.

Once produced, the collagen-containing membrane is functionalized with bioactive molecules such as morphogenetic protein-2 (BMP-2) and zoledronic acid (ZA) and/or nano particles of hydroxyapatite (nHAP) on each side of the membrane.

In one embodiment, nHAP is synthesized using the wet chemical method described in Teotia et al. (2017), ACS Appl. Mater. Interfaces, 9(8), pp 6816-6828. Briefly, an alkaline solution (pH 10.0) of calcium nitrate tetrahydrate (Ca(NO₃)₂.4H₂O, 0.96 M) maintained at 90-100° C. under constant stirring then mixed with aqueous solution of diammonium hydrogen orthophosphate ((NH₄)₂HPO₄, 0.6 M) at a controlled rate. The pH of the system is constantly monitored and maintained at pH 10.0 by adding NH₄OH solution. The nHAP precipitates out of the solution as white crystals. After completion of the reaction, the crystals are maintained in the mother liquor for maturation for 48 h at room temperature in alkaline conditions. After maturation the crystals are filtered out of the solution and washed thoroughly with Milli-Q Type I water (DI-H₂O). The crystals are then dried at 120° C.

In some embodiments, the synthesized nHAP is subjected to thermal treatment to enhance its crystallinity, density, and phase purity. The temperature conditions range from 500 up to 1000° C., with hold time from 1 to 4 h.

The synthesized nHAP is then applied to the collagen-containing membrane simply by soaking the membrane in a sterile saline solution containing the nHAP.

The bioactive molecules may also be incorporated into the nHAP solution at the same time or independently applied to the collagen-containing membrane. The bioactive molecules (ZA, BMP-2) are mixed in sterile water or saline, and then either mixed with dry nHAP using 600 μL of water per gram of nHAP or applied to the collagen-containing membrane by immersion.

The end result of the above process is an artificial periosteum of the present invention.

In one form of the artificial periosteum, a drug-carrier mixture is applied to the functionalized collagen-containing membrane.

Thus, in one form of the artificial periosteum of the present invention there is included a drug-carrier mixture. The carrier component of the drug-carrier mixture may be any calcium-containing carrier mixture known in the art including calcium phosphate cements (CPCs). The carrier component may also comprise other additional carriers.

The therapeutic agents in the drug-carrier mixture of the artificial periosteum of the present invention include bone active agents that are capable of stimulating, promoting, enhancing, or inducing bone formation, or inhibiting bone resorption. The therapeutic agents may be bone-repairing drugs or other bone active agents which alleviate pain and/or inflammation at the treatment site, or treat cancer or treat or prevent a microbial infection. The drug-carrier mixture provides for the release of therapeutic agent at a treatment site. Preferably, the drug-carrier mixture sustains release of the therapeutic agent for prolonged periods of time.

As described below in greater detail, the drug-carrier mixture is prepared by mixing a therapeutic agent with a suitable carrier material such as, for example, a calcium phosphate cement powder. Depending on the particular embodiment, the drug-carrier mixture may be further processed by setting and grinding it into a ground powder. The drug-carrier mixture may be further combined with a suitable bone matrix material, as described below, to form an artificial periosteum of the invention. Alternatively, the drug-carrier mixture may be applied to a functionalized collagen-containing membrane to also form the artificial periosteum of the present invention. The artificial periosteum may then be applied to a treatment site, such as, for example, by implantation.

Calcium Phosphate cements (CPCs) that can be used in the carrier component of the drug-carrier mixture include tri-calcium phosphate mixtures such as α-tri-calcium phosphate (α-TCP) and β-tri-calcium phosphate (β-TCP). Other CPCs that may be used include combinations of dicalcium phosphate and tetracalcium phosphate. Commercially available calcium phosphate cement may also be used such as Hydroset (sold by Stryker Corp), which was used in the Examples disclosed herein. Hydroset is a soft tri-calcium phosphate cement that has the characteristics of a mixture of α-TCP and β-TCP (1:3). In some embodiments the calcium phosphate cements and mixtures thereof may also be seeded with hydroxyapatite (e.g., 2.5% wt./wt. hydroxyapatite crystals). α-TCP and β-TCP may be used in various ratios. For example, in some embodiments the CPC comprises a mixture of α-TCP and β-TCP (1:3) and optionally a seed of hydroxyapatite. In other embodiments, α-TCP and β-TCP may be used in ratios of 1:1 or 1:0. In other embodiments, the CPC is α-TCP cement seeded with 2.5% hydroxyapatite, which produces a harder cement upon setting.

The drug-carrier mixture may include a bone matrix that is at least partially demineralized. The bone matrix may be a demineralized bone matrix putty, or an intact bone matrix that is either partially or wholly demineralized. An intact bone matrix may be used in a bone graft procedure and act as a scaffold for delivery of a bone-repairing drug.

Human demineralized bone matrix putty may also be used in the carrier component of the drug-carrier mixture. It can be obtained from commercial sources such as Puros Demineralized Bone Matrix Putty manufactured by RTI Biologics (Alachua, Fla.). Demineralized bone matrix putty can also be made by the method described by Urist & Dowell (Inductive Substratum for Osteogenesis in Pellets of Particulate Bone Matrix, Clin. Orthop. Relat. Res., 1968, 61, 61-78.). The method involves bone demineralization and defatting and cutting the solid demineralized bone into small pieces that are ground into a course powder under liquid nitrogen. Upon thawing, the ground demineralized bone matrix takes on the consistency of a putty.

If required, setting solutions for tricalcium phosphate cement powders are well known in the art and include solutions of Na₂HPO₄ between 2.5% w/v or commercially available solutions. See Dorozhkin, Materials 2009, 2, 221-291.

Another example of carrier component is one created by combining gelatin with calcium sulphate (CaS) with or without hydroxyapatite (HA) using the cryogelation technology of Kumar et al. (Mater. Today, 13, (2010), 42-44). A further example is described in Raina et al. (2018), J Control Release, Vol. 272, 83-96 using a composite of gelatin-CaS-HA and a similar composite of silk, chitosan, bioactive glass and HA is described in Raina et al.(J Control Release, Vol. 235, 365-378. (2016)). Murphy and co-workers have also described a porous collagen hydroxyapatite-based carrier for delivery of rhBMP-2 and ZA but all lead to cancellous bone regeneration (Murphy et al. (2014), Acta Biomaterialia, Vol 10, Issue 5).

Drug-carrier mixtures can be prepared by dissolving a therapeutic agent in an appropriate solvent such as, for example, ethanol and adding the solution to a carrier mixture. After the solvent has been vented off, the therapeutic agent-carrier mixture is mixed to distribute the therapeutic agent evenly (i.e., homogeneously) throughout the carrier mixture, if required, the therapeutic agent-carrier mixture is then wetted with the appropriate setting solution to produce the drug-carrier mixture.

Methods of Treatment

The artificial periosteum of the present invention is useful in treating bone fracture, and bone loss due to periodontal disease, surgical procedures, cancer, or trauma. Further uses of the artificial periosteum of the invention includes use in increasing bone density in preparation of bone for receiving dental or orthopedic implants, coating of implants for enhanced osseointegration, and use in all forms of spinal fusion.

The present invention provides methods of treatment comprising administering to a patient in need thereof an artificial periosteum of the present invention, which may contain a therapeutically effective amount of a bone-repairing drug, as described herein. The methods of treatment generally include stimulating, promoting, enhancing, or inducing bone formation, or inhibiting bone resorption. The methods of treatment also include, for example, promoting bone remodelling, activating osteoblasts, promoting osteoblast differentiation, inhibiting osteoclasts, increasing the number and activity of osteoblasts, enhancing mean wall thickness, enhancing trabecular bone volume, improving bone architecture, improving trabecular connectivity, increasing cortical thickness, inhibiting bone loss, maintaining/improving bone strength, increasing total bone volume, or volume of the osteoid. The methods of treatment also include treating one or more of osteoporosis, bone fracture, low bone density, or periodontal disease.

In one embodiment of the method of treatment, one or more bone-repairing drugs is administered by release from the drug-carrier mixture as described herein. In another embodiment, a bone-repairing drug is administered from a drug-carrier mixture in combination with another therapeutic agent administered systemically (e.g., orally). For example, a bone-repairing drug may be administered by slow release from an artificial periosteum in combination with one or more additional therapeutic agents to treat bone loss or osteoporosis administered systemically.

The methods of treatment further comprise administration of an artificial periosteum to humans, other mammals, and birds locally to the desired site of action; for example, into a bone void such as a tooth socket defect, adjacent to an alveolar bone, or a bone defect caused by surgery, trauma, or disease.

The invention also provides methods of treating bone-related pain, inflammation, infection and/or bone cancer comprising administering the artificial periosteum containing a therapeutically effective amount of an analgesic, anti-inflammatory agent, an anti-cancer agent, and/or an antimicrobial agent. The methods of treating pain, inflammation, cancer, and/or infection may be combined with any of the foregoing methods of treating bone disorders.

Combination therapy includes administration of a single pharmaceutical dosage formulation containing one or more of the compounds described herein and one or more additional pharmaceutical agents, as well as administration of the compounds and each additional pharmaceutical agent, in its own separate pharmaceutical dosage formulation. For example, a compound described herein and one or more additional pharmaceutical agents, can be administered to the patient together, in an artificial periosteum having a fixed ratio of each active ingredient, or each agent can be administered in separate dosage formulations. For example, a patient may be treated by an artificial periosteum delivering an active drug locally at the site of a bone defect in combination with another drug administered systemically. Where separate dosage formulations are used, the present compounds and one or more additional pharmaceutical agents can be administered at essentially the same time (e.g., concurrently) or at separately staggered times (e.g., sequentially).

In one aspect of the invention, a bone void, for example, is filled with a bone graft or a bone graft substitute derived from natural or synthetics sources, a standalone or a composite substitute, and then covered with the artificial periosteum comprising a functionalized collagen-containing membrane and a drug-carrier mixture of the invention, or with the hydroxyapatite and/or bioactive agent functionalized collagen-containing membrane of the present invention. The collagen-containing membrane acts as a bridge and regenerates bone by means of guided tissue regeneration.

One use of the artificial periosteum of the present invention is as an external covering for defects and/or voids in bones that have been filled with bone graft material. The artificial periosteum may be held in place using any methods known in the art including suturing, clamping or fixed with medical adhesives. The artificial periosteum can also be simply packed under the endosteal bone.

In some embodiments, the artificial periosteum of the present invention is secured in situ using medical adhesive. Medical adhesives have the advantage that they are suitable for contacting bodily fluids. With respect to the artificial periosteum, the medical adhesive can be used to facilitate anchoring the artificial periosteum or could be used to hold together a portion of the artificial periosteum in a structural way. For convenience, adhesive, as used herein, refer generally to the adhesive in a form for application as well as the adhesive composition following curing in a set form. Appropriate medical adhesives should be biocompatible, in that they are non-toxic, non-carcinogenic and do not induce hemolysis or an immunological response. Suitable biocompatible adhesives include commercially available surgical adhesives, such as cyanoacralate (such as 2-octyl cyanoacrylate, DERMABOND™, from Ethicon Products), fibrin glue (such as TISSUCOL® from Baxter) and mixtures thereof, although a wide range of suitable adhesives are available.

For the repair of long segmental defects in bones, the cavity may be filled with any bone graft substitute (synthetic, native, natural), which may or may not include internal or external fixation. Then the artificial periosteum of the present invention can be wrapped around the cortical bone such that the bone graft substitute is held in place so that the two stage Masquelet procedure can be avoided. The Masquelet procedure is used in long bone trauma applications where there is a large intercalary defect, such as where a segment of a long bone is missing. The Masquelet procedure typically comprises two stages: a first stage wherein a spacer is placed and soft tissue forms around the spacer, and a second stage wherein the formed soft tissue is used to cover the bone graft. Thus, in some embodiments, the artificial periosteum of the present invention may be used for trauma repair in a long bone segmental defect. For example, a relatively large covering may be provided with a substance provided therein suitable for trauma repair where the artificial periosteum is used to hold the space (excluding soft tissue) in the long bone and have soft tissue form therearound. The second step of the Masquelet procedure may be avoided because graft materials are provided when the artificial periosteum is originally placed.

A further desirable embodiment includes cells seeded on to or deposited on to the artificial periosteum of the present invention. Any cell may be used, but clearly cells normally associated with promoting growth of bone and bone-associated tissue are preferred. Some preferred examples include, but are not limited to, stem cells, committed stem cells, and differentiated cells including bone marrow stem cells. Other examples of cells used in various embodiments include, but are not limited to, osteoblasts, fibroblasts, chondrocytes, and connective tissue cells.

It will be appreciated that also provided is an artificial periosteum of the present invention for use in such methods of treatment.

It will be appreciated that also provided is use of a functionalized collagen-containing membrane and a drug-carrier mixture in the manufacture if an artificial periosteum for use such methods of treatment.

Definition of Terms

The phrase “therapeutically effective amount” means sufficient amounts of the compounds to treat disorders, at a reasonable benefit/risk ratio applicable to any medical treatment. It is understood, however, that the total dosage of the compounds in the artificial periosteum can be decided by the attending physician within the scope of sound medical judgment. The specific therapeutically effective dose level for any particular patient can depend upon a variety of factors including: the disorder being treated and the severity of the disorder; activity of the specific compound employed; the specific artificial periosteum employed; the rate of drug release from the artificial periosteum; the age, body weight, general health and prior medical history, sex and diet of the patient; the delivery method; drugs used in combination or coincidental with the specific compound employed; and like factors well-known in the medical arts. Actual dosage levels of active ingredients in the pharmaceutical artificial periosteum can be varied so as to obtain an amount of the active compound(s) that is effective to achieve the desired therapeutic response for a particular patient and a particular mode of administration.

The term “bone-repairing drug” or “bone active agent” as used herein refers to an agent that is capable of stimulating, promoting, enhancing, or inducing bone formation, or inhibiting bone resorption. Thus, a bone active agent may be an anabolic drug or an anticatabolic drug. A bone active agent may do one or more of the following: promote bone remodelling, activate osteoblasts, promote osteoblast differentiation, inhibit osteoclasts, increase the number and activity of osteoblasts, enhance mean wall thickness, enhance trabecular bone volume, improve bone architecture, improve trabecular connectivity, increase cortical thickness, inhibit bone loss, maintain/improve bone strength, increase total bone volume, or volume of the osteoid. A bone active agent includes, but is not limited to: prostaglandin E1 (PGE1); prostaglandin E2 (PGE2); an EP2 receptor agonist; an EP4 receptor agonist; an EP2 receptor/EP4 receptor dual agonist; an organic bisphosphonate (e.g., alendronic acid or sodium alendronate); a cathepsin K inhibitor; an estrogen or an estrogen receptor modulator; calcitonin; an inhibitor of osteoclast proton ATPase; an inhibitor of HMG-CoA reductase (i.e., a statin); an αvβ-integrin receptor antagonist; a RANKL inhibitor such as denosumab; a bone anabolic agent, such as parathyroid hormone; a bone morphogenic protein (e.g., BMP-2, BMP-4, BMP-7); Vitamin D or a synthetic Vitamin D analogue such as ED-70; an androgen or an androgen receptor modulator; an activator of Wnt/β-catenin signalling (e.g. a GSK-3 inhibitor, a sclerostin antagonist, a SOST inhibitor); bortezomib; strontium ranelate; platelet-derived growth factor; a pharmaceutically acceptable salt thereof; and a mixture thereof. Bone active agents preferably are not degraded to an inactive form when exposed to a pH of between about 4-5.

The term “calcium phosphate cement” as used herein refers to a bone repair composition that includes a di-calcium phosphate, a tri-calcium phosphate (e.g., α-tri-calcium phosphate and β-tri-calcium phosphate) or a tetra-calcium phosphate, or refers to a bone repair composition that is made from any of the foregoing, or mixtures thereof by setting. A calcium phosphate cement may also include hydroxyapatite incorporated in with a calcium phosphate compound.

The term “drug-carrier mixture” as used herein refers to a mixture of a therapeutic agent incorporated into a calcium carrier component.

The term “agonist” as used herein refers to a compound, the biological effect of which is to mimic the action of the natural agonist. An agonist may have full efficacy (i.e., equivalent to the natural agonist), partial efficacy (lower maximal efficacy compared to the natural agonist), or super maximal efficacy (higher maximal efficacy compared to the natural agonist). An agonist with partial efficacy is referred to as a “partial agonist.” An agonist with super maximal efficacy is referred to as a “super agonist.” In one embodiment, the natural agonist may be PGE2.

Classes of pain relieving agents that may be released from the artificial periosteum include sodium channel blockers (e.g., Nav 1.8 inhibitors, Nav1.9 inhibitors, ropivacaine, bupivacaine, etc.), TRPV1 antagonists, endothelin antagonists (e.g., atrasentan, zibotentan), bradykinin antagonists, ASIC inhibitors, TrkA inhibitors, and radionuclides (⁸⁹Sr, ¹⁵³Sm-lexidronam, ¹⁸⁶Re-etidronate).

Classes of anti-inflammatory agents that may be released from the artificial periosteum include NSAIDS, corticosteroids, and cytokine inhibitors (e.g., inhibitors of TNF-α, IL-1β, etc.).

Classes of antimicrobial agents that may be released from the artificial periosteum include anti-bacterials and antifungals. Anti-bacterials include well-known agents like cephems, cephalosporins, quinolone antibiotics (e.g., ciprofloxacin, levofloxacin, etc.), macrolides (e.g., azithromycin, clarithromycin, erythromycin, etc.). Antifungals include fluconazole, clotrimazole, itraconazole, etc.

Classes of anti-cancer agents that may be released from the artificial periosteum include vincristine, doxorubicin, etoposide, gemcitabine, methotrexate, SRC kinase inhibitors described by Saad in Cancer Treat Rev. 2010, 36(2) 177-84 (e.g., dasatinib, saracatinib, bosutinib).

A bone active agent may be prostaglandin E1, prostaglandin E2, strontium ranelate, calcitonin, parathyroid hormone, Vitamin D, or a synthetic Vitamin D analogue (e.g., ED-70), BMP-2, BMP-4, BMP-7, or platelet-derived growth factor.

A bone active agent may also be an organic bisphosphonate. Organic bisphosphonates include, for example, alendronic acid, sodium alendronate, ibandronate, risedronate, zoledronate, zoledronic acid, etidronate, pamidronate, tiludronate, neridronate, and olpadronate.

A bone active agent may also be a cathepsin K inhibitor including, for example, compounds disclosed and cited by Bromme in Expert Opin. Investig. Drugs 2009, 18(5) 585-600, (e.g., odanacatib).

A bone active agent may be an estrogen or an estrogen receptor modulator including, for example, raloxifene, bazedoxifene, and lasofoxifene, including compounds described at http://en.wikipedia.org/wiki/Selective_estrogen-receptor_modulator.

A bone active agent may be an androgen or an androgen receptor modulator including, for example, testosterone.

A bone active agent may be an inhibitor of osteoclast proton ATPase, including, for example, compounds described by Nyman in Potential of the Osteoclast's Proton Pump as a Drug Target in Osteoporosis, Annales Universitatis Turkuensis 2011, e.g., SB242784, a bafilomycin (e.g., bafilomycin A1), concanamycin A, apicularen, archazolides, benzolactone enamides (salicylihalamide A, lobatamide A), FR167356, FR177995, and diphyllin.

A bone active agent may be an inhibitor of HMG-CoA reductase (i.e., a statin) including, for example those described at http://en.wikipedia.org/wiki/Statin, e.g., atorvastatin, cerivastatin, fluvastatin, lovastatin, mevastatin, pitabastatin, pravastatin, rosuvastatin, and simvastatin.

A bone active agent may be an αvβ-integrin receptor antagonist including, for example, compounds described by Millard et al. in Integrin Targeted Therapeutics, Theranostics 2011, 154-188, e.g., cilengitide (EMD 121974), L000845704, SB2730005.

A bone active agent may be a RANKL inhibitor such as denosumab.

A bone active agent may be an EP2 receptor agonist such as, for example, ONO-AE1-259-01 and CP-533536.

A bone active agent may be an EP2 receptor/EP4 receptor dual agonist such as, for example, those described in Bioorganic & Medicinal Chemistry Letters, 2012, 22(1), 396-401, U.S. Pat. No. 7,402,605, and U.S. Pat. No. 7,608,637. An exemplary EP2 receptor/EP4 receptor dual agonist is 2-((2-((R)-2-((S,E)-3-hydroxy-4-(m-tolyl)but-1-en-1-yl)-5-oxopyrrolidin-1-yl)ethyl)thio)thiazole-4-carboxylic acid (CAS#494223-86-8).

A bone active agent may be an EP4 receptor agonist including, but not limited to, compounds disclosed in U.S. Pat. Nos. 6,043,275, 6,462,081, 6,737,437, 7,169,807, 7,276,531, 7,402,605, 7,419,999, 7,608,637; WO 2002/024647; Bioorganic & Medicinal Chemistry Letters, 2001, 11(15), 2029-2031; Bioorganic & Medicinal Chemistry Letters, 2002, 10(4), 989-1008; Bioorganic & Medicinal Chemistry Letters, 2002, 10(6), 1743-759; Bioorganic & Medicinal Chemistry Letters, 2002, 10(7), 213-2110); Journal of Medicinal Chemistry, 2004, 47(25), 6124-6127; Bioorganic & Medicinal Chemistry Letters, 2005, 15(10), 2523-2526; Bioorganic & Medicinal Chemistry Letters, 2003, 13(6), 1129-1132; Medicinal Chemistry Letters, 2006, 16(7), 1799-1802; Bioorganic & Medicinal Chemistry Letters, 2004, 14(7), 1655-1659; Bioorganic & Medicinal Chemistry Letters, 2003, 13(6), 1129-1132; Journal of Medicinal Chemistry, 1977, 20(10), 1292-1299; Bioorganic & Medicinal Chemistry Letters, 2008, 18(2), 821-824; Bioorganic & Medicinal Chemistry Letters, 2007, 17(15), 4323-4327; Bioorganic & Medicinal Chemistry Letters, 2006, 16(7), 1799-1802; Tetrahedron Letters, 2010, 51(11), 1451-1454; Osteoporosis International, 2007, 18(3), 351-362; Journal of Bone and Mineral Research, 2007, 22(6), 877-888; Heterocycles, 2004, 64, 437-445.

Particular EP4 receptor agonists include, but are not limited to, CP-734432, ONO-4819 (i.e., rivenprost), AE1-329, L-902,688.

In some embodiments, bone active agents included in the artificial periosteum are one or more of alendronic acid, sodium alendronate, ibandronate, risedronate, zoledronate, zoledronic acid, etidronate, pamidronate, tiludronate, neridronate, and olpadronate, odanacatib, raloxifene, bazedoxifene, lasofoxifene, atorvastatin, cerivastatin, fluvastatin, lovastatin, mevastatin, pitabastatin, pravastatin, rosuvastatin, simvastatin, strontium ranelate, calcitonin, parathyroid hormone, or bone morphogenic protein-2.

In other embodiments, bone active agents included in the artificial periosteum are one or more of an EP2 receptor agonist, an EP2 receptor/EP4 receptor dual agonist, an EP4 receptor agonist, an organic bisphosphonate, an estrogen receptor modulator, an inhibitor of HMG-CoA reductase, and strontium ranelate.

The invention also provides for artificial periosteum that includes a combination of any of the agents, drugs or classes of drugs described herein. For example, one or more agents/drugs that activate osteoblasts may be combined with one or more agents/drugs that inhibit osteoclasts.

Alternatively, multiple agents/drugs that either activate osteoblasts or inhibit osteoclasts may be combined.

In some embodiments, artificial periosteum may include an EP4 receptor agonist with any one or more of the following: a bisphosphonate; a cathepsin K inhibitor; an estrogen or an estrogen receptor modulator; calcitonin; an inhibitor of osteoclast proton ATPase; an inhibitor of HMG-CoA reductase (i.e., a statin); an αvβ3-integrin receptor antagonist; a RANKL inhibitor such as denosumab; a bone anabolic agent, such as parathyroid hormone; a bone morphogenic protein (e.g., BMP-2, BMP-4, BMP-7); Vitamin D or a synthetic Vitamin D analogue such as ED-70; an androgen or an androgen receptor modulator; an activator of Wnt/β-catenin signalling (e.g. a GSK-3 inhibitor, a sclerostin antagonist, a SOST inhibitor); bortezomib; strontium ranelate; platelet-derived growth factor.

In some embodiments, for example, an EP4 receptor agonist is combined with one or more bisphosphonates selected from alendronic acid, sodium alendronate, ibandronate, risedronate, zoledronate, zoledronic acid, etidronate, pamidronate, tiludronate, neridronate, and olpadronate.

In other embodiments, an EP4 receptor agonist is combined with one or more of raloxifene, bazedoxifene, and lasofoxifene.

In other embodiments, an EP4 receptor agonist is combined with a bone morphogenic protein, e.g., BMP-2, BMP-4, or BMP-7. For example, one combination includes CP-734432 with either BMP-2 or BMP-7. Another combination includes ONO-4819 (rivenprost) with BMP-2 or BMP-7. Yet another combination includes AE1-329 with BMP-2 or BMP-7. Still another combination includes L-902,688 with BMP-2 or BMP-7. A further combination includes 7-((R)-3,3-difluoro-5-((3S,4S,E)-3-hydroxy-4-methylnon-1-en-6-yn-1-yl)-2-oxopyrrolidin-1-yl)heptanoic acid with BMP-2 or BMP-7. Another combination includes 7-((R)-2-((3S,4S,E)-3-hydroxy-4-methylnon-1-en-6-yn-1-yl)-5-oxopyrrolidin-1-yl)heptanoic acid with BMP-2 or BMP-7.

In still other embodiments, an EP4 receptor agonist is combined with a statin such as, for example, atorvastatin, cerivastatin, fluvastatin, lovastatin, mevastatin, pitabastatin, pravastatin, rosuvastatin, and simvastatin.

All patents, patent applications, provisional applications, and publications referred to or cited herein are incorporated by reference in their entirety, including all figures and tables, to the extent they are not inconsistent with the explicit teachings of this specification.

It is to be understood that if any prior art publication is referred to herein, such reference does not constitute an admission that the publication forms a part of the common general knowledge in the art in Australia or any other country.

Following are examples that illustrate procedures for practising the invention. These examples should not be construed as limiting.

### EXAMPLES

### Example 1

### In Vivo Study

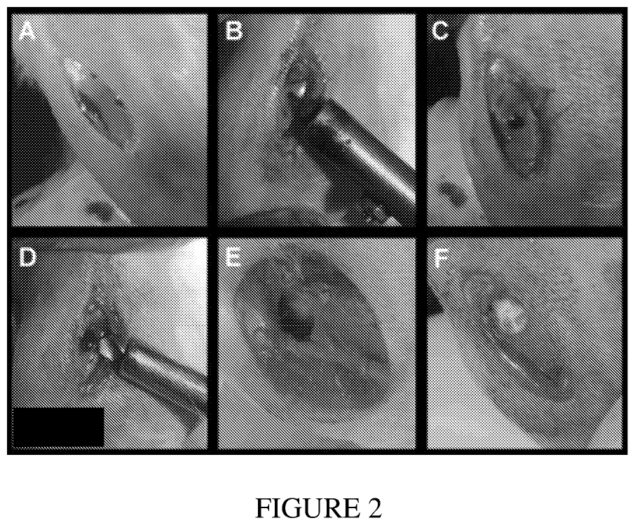

In a first study, we used a tibia defect model previously described by Horstmann et al. Briefly, a 4.5 mm defect was created in the proximal tibia of Sprague-Dawley male rats by drilling the cortex as well as the metaphyseal bone underneath. FIG. 2 shows one example of a surgical procedure of the tibia defect model. The defects were filled using a gelatin-calcium sulphate-hydroxyapatite scaffold as one of the available bone void fillers in the cancellous cavity with or without ZA and rhBMP-2. As described in Raina et al. (2018), J Control Release, Vol. 272. This was done to provide a support to the overlaying collagen-containing membrane, which otherwise would have been difficult to place. In some of the groups, the scaffolds were also covered with a 6 mm piece of collagen membrane and the remaining membrane was inserted into the endosteum. In this way, the membrane prevented the scaffold from leaking outside of the circular defect. A thorough description of the groups and the doses of different bioactive molecules is given in Table 1.

TABLE 1
Groups, doses and sample size in the tibia defect study.

| | | Sample |
|---|---|---|
| | | Size |
| Group | Treatment | (n) |
| 1 | S | 10 |
| 2 | S + ZA (10 μg) + (CM) | 10 |
| 3 | S + ZA (10 μg) + BMP (5 μg) + (CM) | 12 |
| 4 | S + ZA (10 μg) + (CM + BMP-2 (2 μg)) | 10 |
| 5 | S + ZA (10 μg) + BMP-2 (3 μg) + | |
| | (CM + BMP-2 (2 μg)) | 10 |

S = Gelatin-calcium sulphate-hydroxyapatite scaffold,
ZA = Zoledronic acid,
CM = Collagen membrane,
BMP-2 = Bone morphogenic protein-2

The animals were sacrificed 8 weeks post-surgery and were subjected to quantitative micro-CT and representative histology for evaluation of defect healing.

In a second study, collagen membrane (4 mm circular piece) alone or functionalized with 1 mg of nanoparticles of hydroxyapatite on each side of the membrane were analyzed in the abdominal muscle pouch model, previously described by Raina et al. and the following groups were used:

1. Collagen membrane (CM) alone,

2. CM+nanoparticles of hydroxyapatite (nHA),

3. CM+rhBMP-2 (10 μg),

4. CM+nHA+rhBMP-2 (10 μg),

5. CM+rhBMP-2 (10 μg)+ZA (10 μg),

6. CM+nHA+rhBMP-2 (10 μg)+ZA (10 μg).

A total of n=5/group was used and the animals were sacrificed 4 weeks post-surgery followed by X-ray radiography and micro-CT quantifications.

Results

Study 1: Micro CT

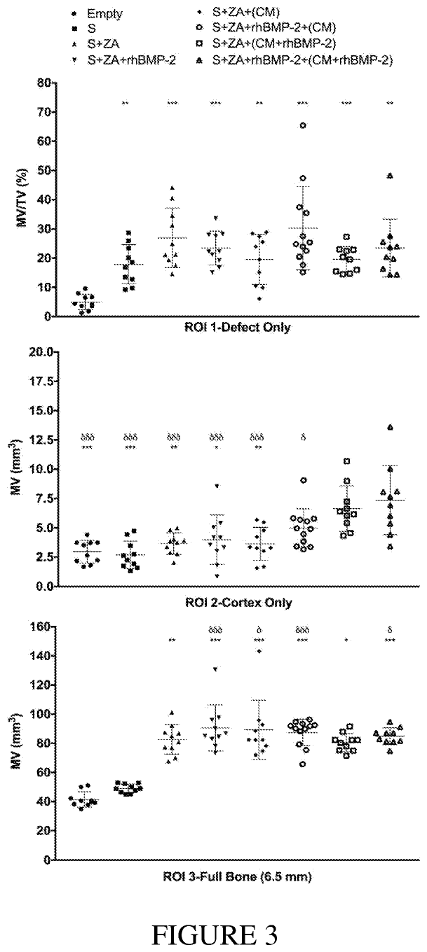

Region of interest 1 (ROI 1): For the micro-CT analysis, we had defined 3 ROI's. ROI 1 measured the Mineralized Volume (MV)/Tissue Volume (TV) % within the defect hole excluding the cortex. The diameter of the ROI 1 was dynamic and thus measured 4.5 mm at the top and narrowed down to 1.5 mm in the bottom. The depth of the ROI 1 was 2 mm. The micro-CT measurements revealed that all scaffold and membrane treated group irrespective of the bioactive molecule, regenerated significantly higher volume of mineralized tissue or MV/TV % as compared to the empty group (FIG. 3, Top).

Region of interest 2 (ROI 2): For assessment of cortical healing, we used the measurements from ROI 2. ROI 2 consisted of a 4.5 mm circular ROI, which extended upwards from the bottom of the old cortex. As of consequence of this, we measured the bone regenerated in the areas of the regenerated cortex, which was previously taken away during surgery. S+ZA+rhBMP-2+(CM) (group 6) and S+ZA+(CM+rhBMP-2) group (group 7) had significantly higher cortical mineralized volume (MV) when compared to groups the empty group (group 1) or the scaffold only group (group 2). (For p-values, refer to FIG. 3, Middle.)

FIG. 3 shows the micro-CT quantifications of the tibia defect study 8-weeks post surgery. * In top graph indicates comparison of respective groups with the empty group. * In the middle graph indicates p values of all groups compared to the S+ZA+(CM+rhBMP-2) group. δ Indicates comparison of the S+ZA+rhBMP-2+(CM+rhBMP-2) vs. all other groups. * In the bottom group indicates comparison of respective groups with the empty group while δ indicates the comparison of the respective groups with the scaffold only group. * or δ indicates p<0.05, ** or δδ indicates p<0.01 and *** or δδδ indicates p<0.001.

Region of interest 3 (ROI 3): The last ROI, ROI 3 was used to measure the full defect as well as the bone proximal and distal to the defect. This not only provides a measure of the bone regenerated in the defect area but also provide an insight into the volume of mineralized tissue regenerated around the implanted scaffold and membrane. ROI 3 was 6.5 mm in height covering the 4.5 mm defect and 1 mm proximal and 1 mm distal areas of the defect. The results (FIG. 3, Bottom) indicated that groups 3-8 had significantly higher MV compared to group 1. Furthermore, groups 4, 5, 6 and 8 had significantly higher MV compared to group 2 also. No significant differences were seen between group 1 and 2 or between any of the groups 3-8.

Study 1: Cortical Healing Via Micro-CT

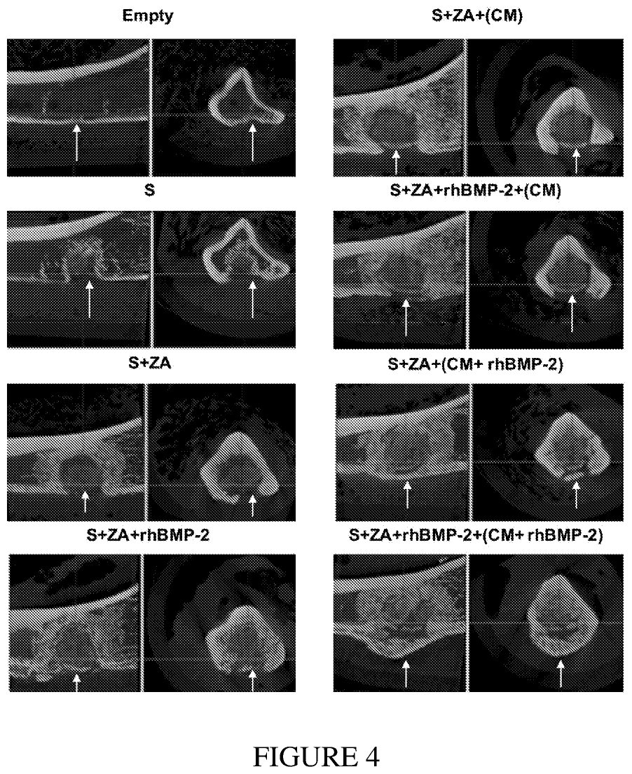

FIG. 4 shows the evaluation of cortical healing using micro-CT. White arrows point at the cortical location of the defect and the extent of cortical regeneration (images for representation only).

Almost all samples in the empty group had healed on the cortical side with very thin cortical bone at the defect location. Little or no bone formation could be seen within the defect. Only scaffold treated groups (2-4) had varying degree of bone formation within the defect but did not lead to cortical regeneration. In groups 5 and 6, a white radio dense rim could be seen along the surface of the membrane, which also confirmed the endosteal placement of the membrane. All membrane treated groups (5-8) confined the scaffold within the defect. Group 7 (5/10) and group 8 (7/10) significantly improved the cortical bridging and were the only groups with maximum cortical bridging among groups treated with the scaffold or the membrane. Refer to FIG. 4 for more details.

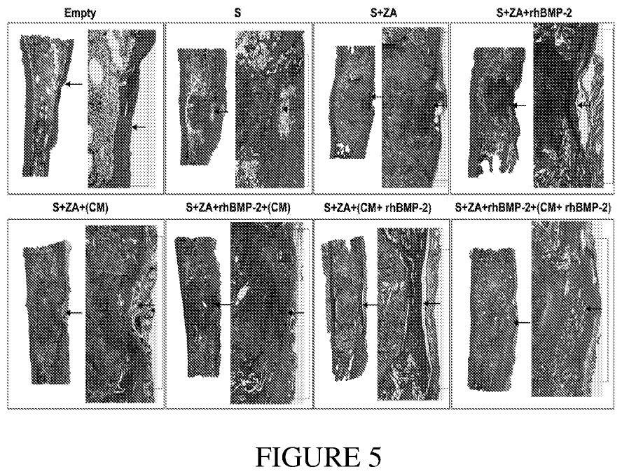

In FIG. 5, the images on the left provide a low magnification overview of defect healing while the images on the right provide a high magnification view of cortical healing. The box indicates the extent of cortical defect and the black arrow is placed approximately in the middle of the cortical defect.

Histology results corroborated well with the micro-CT imaging. Empty group showed a thin but healed cortex and is infiltrated with marrow tissue in the metaphyseal zone. Group 2, scaffold showed some bone formation in the periphery of the scaffold but no cortical healing. Groups 3-6 showed significant amounts of new trabecular bone around the defect and some bone formed within the scaffold pores as well. However, only some cortical bone regeneration was seen. Representative histology images show cortical bridging in groups 7 & 8. The inside of the defect, similar to groups 3-6 was filled with trabecular bone on the periphery of the scaffold but limited bone had formed within the scaffold.

Study 2: X-Ray Radiography

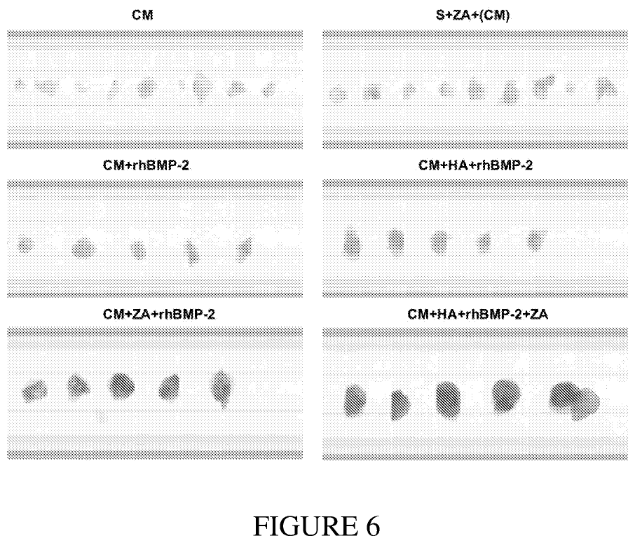

The radiographs shown in FIG. 6 indicate that the addition of rhBMP-2 to the collagen-containing membrane with or without hydroxyapatite led to an increase in the radio density of the specimens when compared to the collagen-containing membrane alone. Addition of both ZA and rhBMP-2 to the collagen-containing membrane with or without nHA increased the radio density of the specimens significantly.

Conclusions

Study 2 demonstrated the true carrier property of the collagen-containing membrane by inducing bone in the abdominal muscle pouch model. Delivery of both rhBMP-2 and rhBMP-2+ZA irrespective of the presence of nHA induced bone formation to varying degrees and co-delivery of both rhBMP-2 and ZA induced higher bone than in rhBMP-2 group. Addition of nHA to the collagen membrane further increased the bone forming potential of the collagen membrane when both rhBMP-2 and ZA were delivered using the membrane. No such effect was seen when only rhBMP-2 was added.

Study 1 elucidated the true potential of the membrane in cortical healing via the phenomenon of guided tissue regeneration. Apart from the empty group, groups 2-4 failed to show a complete cortical regeneration. Addition of a membrane on top of the scaffolds in groups 5-8 prevented the scaffolds from being forced out of the defect and blocking cortical regeneration. This study also showed that delivery of ultra-low doses of rhBMP-2 via the collagen-containing membrane significantly increased cortical regeneration as seen in both groups 7 and 8. The membrane functionalized with low doses of rhBMP-2 can thus be used to regenerate bone in demanding orthopedic situations.

### Example 2

### Functionalized Collagen-Containing Membrane

As discussed in the specification above, an artificial periosteum of the present invention, i.e. one comprising a hydroxyapatite functionalized collagen-containing membrane as described herein, can be used as a containment device to protect biomaterials that are filled in a bone void from leaking out into the cortical bone. We believe that when a biomaterial (ceramic or polymeric) is placed in a bone defect, it tends to get pushed outside of the bone, most likely due to a hydrostatic pressure built up within the bone. This phenomenon most likely causes impaired cortical bone healing. However, when the artificial periosteum of the present invention is used, especially endosteally, to cover the biomaterial placed in the bone void, i.e. under the inside ends of the cortical bone (endosteum), it prevents the biomaterial from being pushed out into the cortical bone. While not essential, we believe that the endosteal placement of the artificial periosteum is important because it provides a firm grip to the artificial periosteum to cover the implanted material throughout the duration of the experiment.

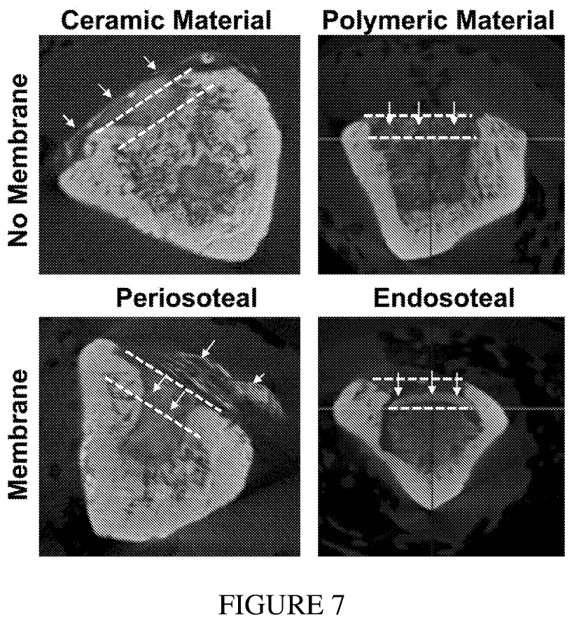

FIG. 7 shows the role of artificial periosteum of the present invention as a containment device for ceramic or polymeric biomaterials placed within a bone void. Dashed lines indicate inner and outer margins of the cortical bone. Arrows in top left and top right indicate the ceramic and the polymeric biomaterial, respectively, protruding out and sitting between the cortical ends, as indicated by the lower dashed line. Bottom arrows in the left bottom panel shows the ceramic material leaking into the cortical location of the bone, while upper arrows indicate mineralization of the collagen membrane, which was placed periosteally. Arrows in bottom right point at the artificial periosteum covering the polymer scaffold placed in the bone defect. Note that the membrane has mineralized to some extent and also ensured that the material always remained under the cortical bone and not between the cortical ends. All images are representative micro-CT slices taken after 8 weeks of in vivo treatment.

Of course, the other role of the artificial periosteum is to act as a bridge and regenerate cortical bone by means of guided tissue regeneration (see FIG. 4). The experiments conducted have shown that when the membrane is placed periosteally, since it is not securely covering the defect hole, it gets pushed up from the cortex and tends to mineralize in the overlaying muscle. However, endosteal placement of membrane with or without rhBMP-2 has shown both containment of the biomaterial in the bone void as well as cortical bone regeneration (more so with small doses of rhBMP-2 on the membrane).

### EXAMPLE 3

### Comparative Study

We used the published abdominal muscle pouch model (Raina et al. (2018), J. Control Release, Volume 272 Pages 83-96) to compare the commercially available ACS collagen sponge (Medtronic) with BMP-2 and ZA to compare with the functionalized collagen-containing membrane of the present invention.

We took micro-CT data from the ACS groups and compared it with data on the artificial periosteum in the muscle pouch.

Both studies have been done in the abdominal muscle pouch model with the same dose rhBMP-2 (10 micro gram/scaffold) and ZA (10 micro gram/scaffold). While direct comparison of these data is not entirely possible since experiments have been carried out at two different time points and micro-CT phantom calibration is not available, we did note that the same micro-CT settings were used and the voxel size was the same (10 microns). Furthermore, X-rays were taken at the same settings so they also show differences.

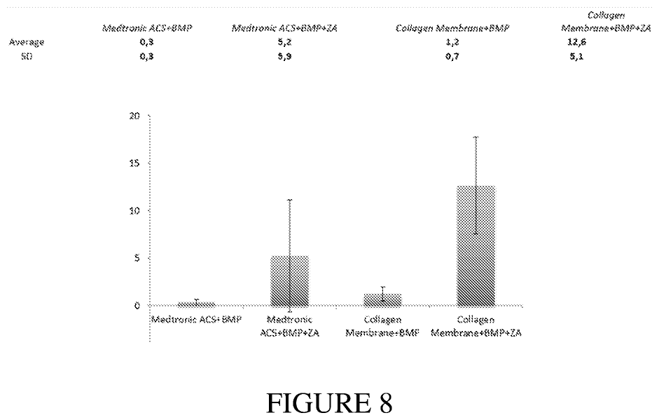

We found that the artificial periosteum was superior to the ACS groups in the formation of bone (FIG. 8).

Concluding Statements

In view of the above, the present invention has a number of advantages over the prior art such as:
Ease of use in obviating the need to obtain bone grafts of the appropriate size and shape, graft-site morbidity, and tissue integration with the surrounding tissue; and
Providing an alternative bone repair method especially the development of an artificial periosteum which could also be used to locally deliver bone active agents like BMP-2 over extended periods of time to promote bone growth and repair bone defects.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

It is to be understood that the invention is not limited to specific features shown or described since the means herein described comprises preferred forms of putting the invention into effect. The invention is, therefore, able to be claimed in any of its forms or modifications.

1. An artificial periosteum comprising a functionalized collagen-containing membrane and a drug-carrier mixture, wherein the drug-carrier mixture comprises at least one therapeutic agent and a calcium-containing carrier mixture.

2. An artificial periosteum according to claim 1, wherein the functionalized collagen-containing membrane is a hydroxyapatite functionalized collagen-containing membrane.

3. An artificial periosteum according to claim 1, wherein the drug-carrier mixture comprises a bone active agent.

4. An artificial periosteum according to claim 3, wherein the bone active agent activates osteoblasts.

5. An artificial periosteum according to claim 3, wherein the bone active agent inhibits osteoclasts.

6. An artificial periosteum according to claim 3, wherein the bone active agent comprises one or more of: PGE1; PGE2; an EP2 receptor agonist; EP4 receptor agonist; an EP2 receptor/EP4 receptor dual agonist; an organic bisphosphonate; a cathepsin K inhibitor; an estrogen or an estrogen receptor modulator; calcitonin; an inhibitor of osteoclast proton ATPase; an inhibitor of HMG-CoA reductase; an integrin receptor antagonist; a RANKL inhibitor; a bone anabolic agent; a bone morphogenetic agent; Vitamin D or a synthetic Vitamin D analogue; an androgen or an androgen receptor modulator; a SOST inhibitor; platelet-derived growth factor; a pharmaceutically acceptable salt thereof; and a mixture thereof.

7. An artificial periosteum according to claim 6, wherein the organic bisphosphonate is selected from the group consisting of alendronic acid, sodium alendronate, ibandronate, risedronate, zoledronate, zoledronic acid, etidronate, pamidronate, tiludronate, neridronate, and olpadronate.

8. An artificial periosteum according to claim 6, wherein the bone morphogenic protein is selected from the group consisting of BMP-2, BMP-4 and BMP-7.

9. An artificial periosteum according to claim 1, wherein the drug-carrier mixture comprises a sodium channel blocker, a TRPV1 antagonist, an endothelin antagonist, a bradykinin antagonist, an ASIC inhibitor, a TrkA inhibitor, or a radionuclide.

10. An artificial periosteum according to claim 1, wherein the drug-carrier mixture comprises an anti-inflammatory agent selected from the group consisting of a NSAID, a corticosteroid, and a cytokine inhibitor.

11. An artificial periosteum according to claim 1, wherein the drug-carrier mixture comprises an anti-bacterial agent and/or an antifungal agent.

12. An artificial periosteum according to claim 11, wherein the anti-bacterial agent is a cephem, a cephalosporin, a quinolone antibiotic and/or a macrolide.

13. An artificial periosteum according to claim 11, wherein the antifungal agent is fluconazole, clotrimazole and/or itraconazole.

14. An artificial periosteum according to claim 1, wherein the drug-carrier mixture comprises an anti-cancer agent.

15. An artificial periosteum according to claim 14, wherein the anti-cancer agent is vincristine, doxorubicin, etoposide, gemcitabine and/or methotrexate.

16. An artificial periosteum comprising a hydroxyapatite functionalized collagen-containing membrane and a drug-carrier mixture, wherein the drug-carrier mixture comprises BMP-2 and zoledronic acid

17. A method of repairing bone, comprising the step of implanting an artificial periosteum according to claim 1 into a bone defect.

18.-32. (canceled)

33. An artificial periosteum according to claim 16, wherein the functionalized collagen-containing membrane is a hydroxyapatite functionalized collagen-containing membrane.

34. A method of repairing a bone defect, comprising the step of:
(i) implanting into said bone defect a graft material; and
(ii) covering said graft with a functionalized collagen-containing membrane.

35. A method of repairing bone, comprising the step of implanting an artificial periosteum according to claim 16 into a bone defect.
